(19) United States
(12) Patent Application Publication
(10) Pub. No.: US 2023/0404919 A1
(43) Pub. Date: Dec. 21, 2023

(54) INTRINSICALLY-DISORDERED PROTEINS AS EMULSIFIERS

(71) Applicant: THE JOHNS HOPKINS UNIVERSITY, Baltimore, MD (US)

(72) Inventors: Geraldine Seydoux, Baltimore, MD (US); Andrea Putnam, Baltimore, MD (US); Andrew Folkmann, Baltimore, MD (US)

(21) Appl. No.: 18/249,676

(22) PCT Filed: Oct. 21, 2021
(86) PCT No.: PCT/US21/55990

§ 371 Date: Apr. 19, 2023

Related U.S. Application Data
(60) Provisional application No. 63/094,987, filed on Oct. 22, 2020.

(51) Int. Cl. A61K 9/107; A61K 47/42; A61K 8/64; A61K 8/06; A61Q 19/00; A61K 47/36; A61K 8/60; A01N 25/04; A23L 29/10

(52) U.S. Cl. CPC A61K 9/107; A61K 47/42; A61K 8/64; A61K 8/06; A61Q 19/00; A61K 47/36; A61K 8/606; A01N 25/04; A23L 29/10; A61K 2800/10

(57) ABSTRACT

Described are emulsions and emulsion systems comprising intrinsically-disordered proteins (e.g. MEG proteins) as stabilizers for protein rich compositions.

### CROSS-REFERENCE TO RELATED APPLICATIONS

This application claims the benefit of U.S. Provisional Application No. 63/094,987 filed Oct. 22, 2020, the contents of which is herein incorporated by reference in their entirety.

### TECHNICAL FIELD

The present disclosure relates to emulsions and emulsion systems comprising intrinsically-disordered proteins as stabilizers.

### BACKGROUND OF THE INVENTION

Emulsions are mixtures of two or more immiscible liquids where one liquid forms droplets (dispersed phase) suspended in the other liquid (continuous phase). Emulsions are used in a wide range of applications, including food science, cosmetics and pharmacology. Examples include milk, an emulsion of fat droplets dispersed in water.

Emulsions are inherently unstable and decay over time by coalescence and Ostwald ripening. If left to settle, raw milk will separate into distinct cream and whey (watery liquid) layers. This decay or “coarsening” is due to the high energy state of molecules at the interface between the two phases. The high energy of the interface drives the system to reduce the total interfacial area, by reducing the number of droplets and increasing their size. This is achieved by droplets fusing with each other (coalescence) and small droplets losing material to large droplets (Otswald ripening). Eventually, the two liquids in the emulsion are fully separated and the surface area between the two liquids is fully minimized.

Agents that stabilize emulsions (emulsifiers) against coalescence and Otswald ripening are of great value to increase the shelf-life of emulsions. One type of agent that is commonly used are nanoscale solid particles that have the property of being wetted by both liquids. These particles adsorb strongly at the interface between the two liquids and lower the free energy of molecules at the interface. The energy required to remove the adsorbed particles exceeds the energy gained by reducing interfacial surface area, which suppresses Ostwald ripening. The particles also present a physical barrier to coalescence. These types of particles are called Pickering stabilizers (or Ramsden stabilizers) for the two scientists who first described this phenomenon over 100 years ago. Emulsions stabilized by Pickering stabilizers are often referred to as Pickering emulsions. Homogenized milk is an example of an oil-in-water (o/w) Pickering emulsion where fat droplets are stabilized by globules of casein, a protein naturally secreted in milk. Much research has been invested in discovering and characterizing new additives that can function as Pickering stabilizers for the wide-range of emulsions used in food science, cosmetics, pharmacology and other fields (Berton-Carabin and Schroen, 2015).

### SUMMARY OF THE INVENTION

The present invention is directed to an emulsion system comprising: an intrinsically disordered protein selected from the group consisting of MEG (maternal-effect germline defective) proteins or a combination thereof and a protein in need of stabilization exogenous to the intrinsically disordered protein. In some embodiments, the emulsion system further comprises a single-stranded nucleic acid (e.g. RNA). In some embodiments, the intrinsically disordered protein comprises MEG-3. In some embodiments, the emulsion system comprises a water-in-water emulsion.

The present invention is also directed to compositions comprising the emulsion system described herein.

Other aspects of the invention will become apparent by consideration of the detailed description and accompanying drawings.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1C shows MEG-3 forms non-dynamic assemblies adsorbed on the surface of liquid PGL condensates. FIG. 1A is photomicrographs of condensates formed at indicated concentrations of MEG-3 trace-labeled with Alexa647 (green) in the presence of 20 ng/uL nos-2 RNA trace-labeled with Alexa546 (magenta) and incubated for 30 min. FIG. 1B is representative gel sedimentation assays of condensate reactions assembled as in FIG. 1A. Samples of the reaction before sedimentation (total) and fractions after sedimentation (supernatant, pellet) were resolved by SDS-PAGE and stained with SimplyBlue™ SafeStain. FIG. 1C is photomicrographs of MEG-3 clusters (pink) adsorbed on the surface of liquid droplets formed by the protein PGL-3 (green). Challenge with high salt (1M NaCl) caused dispersion of the liquid phase. Empty MEG-3 “spheres” remain.

FIGS. 2A-2F shows MEG-3 reduces coarsening of PGL-3 emulsions. FIG. 2A is photomicrographs of maximum projections of one cell C. elegans embryos expressing mCherry-PGL-3.

FIG. 2B is photomicrographs of maximum projections of one cell embryos in the presence of MEG-3.

FIG. 2C is histograms of PGL condensate volumes measured by IMARIS from images as in A) and B). Circles indicate the fraction of volume of PGL-3 in condensates binned by the radius of each condensate in the absence of MEG-3 (black) or in the presence of MEG-3 (green). Error bars represent the SEM. Lines indicate fit of data to a lognormal distribution. FIG. 2D is a graph of the total number of droplets in the presence or absence of MEG-3. FIG. 2E is a graph the total volume of PGL in droplets in the presence or absence of MEG-3. Each point represents an embryo. Line represents the mean. FIG. 2F is a graph of the total surface area of PGL droplets in the presence or absence of MEG-3. Each point represents an embryo. Line represents the mean.

FIGS. 3A-3G shows MEG-3 prevented coarsening of PGL-3 emulsions in vitro. FIG. 3A is photomicrographs of a protein-rich condensates of 3 μM PGL-3 (green, trace labeled with Dylight-488) and 80 ng/μL nos-2 RNA with or without 500 nM MEG at indicated time points after initiation of condensation. Scale bar is 5 μm. FIG. 3B is histograms of PGL condensates assembled as in FIG. 3A without MEG-3. Circles indicate the fraction of volume of PGL-3 in condensates binned by the radius of each condensate. Lines indicate fit of data to a lognormal distribution. FIG. 3C is histograms of PGL condensates with 500 nM MEG-3. FIG. 3D is a graph showing the total volume of PGL-3 in droplets at indicated timepoints without (black) or with 500 nM MEG-3 (green). FIG. 3E is a graph showing the total surface area of PGL-3 in droplets without (black) or with 500 nM MEG-3 (green). FIG. 3F is a graph of the number of PGL-3 droplets at indicated timepoints without (black) or with 500 nM MEG-3 (green). FIG. 3G is a graph of the mean radius of PGL-3 droplets at indicated timepoints without (black) or with 500 nM MEG-3 (green) calculated from data fitting of histograms shown in FIG. 3B and FIG. 3C.

### DETAILED DESCRIPTION OF THE INVENTION

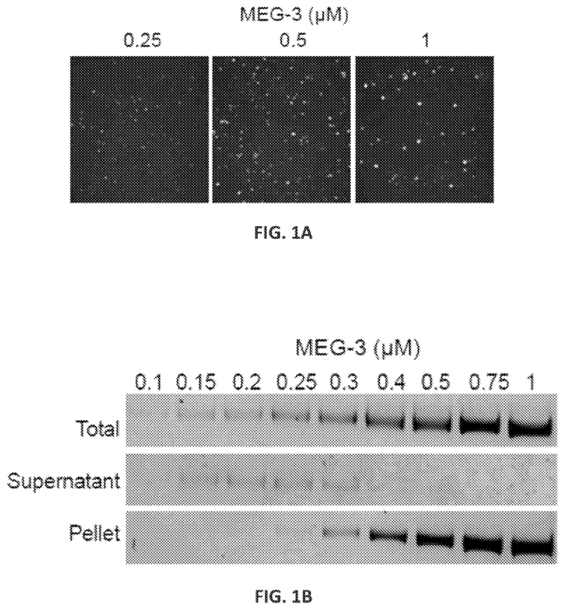

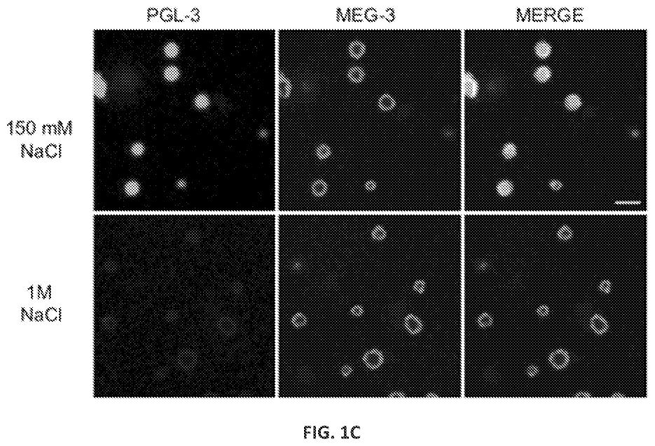

Disclosed herein is are emulsions stabilized by naturally-occurring proteins that do not adopt a specific ordered globular structure. The intrinsically-disordered proteins, MEG proteins, from the nematode C. elegans, function as Pickering stabilizers for water-in-water (w/w) emulsions. MEG-3 forms nanoscale solid clusters that adsorb onto the surface of liquid condensates (FIG. 1). Addition of RNA to purified recombinant MEG-3 leads to the formation of MEG-3/RNA clusters able to stabilize a w/w emulsion in a reconstituted in vitro system. Unlike previously described Pickering stabilizers, the MEG proteins are natural, organic, renewable agents that can be produced outside of cells to stabilize artificial emulsions when combined with RNA.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10/a of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Cell,” as used herein, refers to the basic functional unit of life, and includes both prokaryotic and eukaryotic cells. Cells are characterized by an interior having the nucleus or nucleoid, and a cell membrane (cell surface). Cells can also have a cell wall. Cells without a cell wall include eukaryotic cells, mammalian cells, and stem cells. Cells with a cell wall include prokaryotic cells and plant cells. Other cells are useful in the present invention. In some embodiments, the cell is a mammalian cell.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide”, “protein,” and “peptide” are used interchangeably herein.

Any compositions or methods provided herein can be combined with one or more of any of the other compositions and methods provided herein.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### 2. EMULSIONS OR EMULSION SYSTEMS

The present disclosure provides emulsion systems comprising an intrinsically disordered protein and a protein in need of stabilization exogenous to the intrinsically disordered protein. The protein in need of stabilization may be an inherently water insoluble protein or include proteins that become insoluble at increased concentration, such as in protein-rich composition. In some embodiments, the emulsion system comprises a water-in-water emulsion.

In some embodiments, the emulsion system is for use with protein-rich compositions.

Intrinsically disordered proteins (IDPs) lack stable tertiary or secondary structures and exist as populations of quickly interconverting conformations that resemble the denatured states of ordered proteins. IDPs were originally identified and characterized by biochemical and spectroscopic methods, but can be readily identified by sequence analysis, as known in the art, because of their biased amino acid composition and, in particular, their low content of hydrophobic residues, which prevents them from folding spontaneously.

In some embodiments, the intrinsically disordered protein is selected from the group consisting of MEG (maternal-effect germline defective) proteins or a combination thereof. In some embodiments, the intrinsically disordered protein comprises MEG-3. In some embodiments, the intrinsically disordered protein comprises MEG-4. In certain embodiments, the intrinsically disordered protein comprises MEG-3 and MEG-4.

In some embodiments, the emulsion system further comprises a single-stranded nucleic acid. The single-stranded nucleic acid may associate with or bind to the intrinsically disordered protein. In some embodiments, the single-stranded nucleic acid comprises RNA (e.g., an RNA or an DNA/RNA hybrid). In some embodiments, the single-stranded nucleic acid comprises DNA.

The single-stranded nucleic acid comprises between 20 and 5000 nucleotides in length. In some embodiments, the single-stranded nucleic acid comprises about 200 and 2000 nucleotides. In some embodiments, the single-stranded nucleic acid comprises between 500 and 750 nucleotides.

The molar ratio of the single-stranded nucleic acid to the intrinsically disordered protein may be about 0.1-1:1. For example, the molar ratio may be 0.1-1, 0.1-0.8, 0.1-0.6, 0.1-0.4, 0.1-0.2, 0.2-1.1, 0.2-1, 0.2-0.8, 0.2-0.6, 0.2-0.4, 0.4-1.1, 0.4-1, 0.4-0.8, 0.4-0.6, 0.6-1.1, 0.6-1, 0.6-0.8, 0.8-1.1, 0.8-1, or 1-1.1. In some embodiments, the molar ratio of the single-stranded nucleic acid to the intrinsically disordered protein may be about 0.5:1.

Alternatively, in some embodiments, the emulsion system further comprises a non-nucleic acid negatively charged polymer (e.g., a polyanion) in place of, or in addition to, the single-stranded nucleic acid. Exemplary polyanions include natural polymers such as hyaluronic acid, alginate, dextran sulfate, carrageenan, chondroitin sulfate, pectin, xanthan gum, cellulose, collagen, heparin, or synthetic polymers such as polyacrylates.

The mole or molar ratio of the protein in need of stabilization and the intrinsically disordered protein may be at least 2. In some embodiments, the mole or molar ratio of the protein in need of stabilization and the intrinsically disordered protein may be about 2 to 20 (about 2, about 4, about 6, about 8, about 10, about 12, about 15, about 18 or about 20). In some embodiments, the mole or molar ratio may be about 4-10, 4-15, 4-20, 6-10, 6-15, 6-20, 12-15, 12-20, or 15-20.

The present disclosure also provides a composition comprising the emulsion system disclosed herein. The composition may be a cosmetic composition, a pharmaceutical or nutraceutical, a pesticide or insecticide composition, a fertilizer, a food or drink, nanomaterials, oil and gas recovery, and the like.

The composition may comprise additional components, including but not limited to, an active agent, additional proteins, diluents, lubricants, binders, disintegrants, colorants, flavors, sweeteners, antioxidants, preservatives, glidants, solvents, suspending agents, wetting agents, surfactants, emollients, combinations thereof, and others. Amounts of the additional components in the compositions may vary depending on the type of composition prepared.

Suitable diluents include sugars such as glucose, lactose, dextrose, and sucrose; diols such as propylene glycol; calcium carbonate; sodium carbonate; sugar alcohols, such as glycerin; mannitol; and sorbitol.

Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of Theobroma.

Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose.

Suitable disintegrants include agar, alginic acid and the sodium salt thereof, effervescent mixtures, croscarmelose, crospovidone, sodium carboxymethyl starch, sodium starch glycolate, clays, and ion exchange resins.

Suitable colorants include a colorant such as an FD&C dye.

Suitable flavors include menthol, peppermint, and fruit flavors.

Suitable sweeteners include aspartame and saccharin.

Suitable antioxidants include butylated hydroxyanisole (“BHA”), butylated hydroxytoluene (“BHT”), and vitamin E.

Suitable preservatives include benzalkonium chloride, methyl paraben and sodium benzoate.

Suitable glidants include silicon dioxide.

Suitable solvents include water, isotonic saline, ethyl oleate, glycerine, hydroxylated castor oils, alcohols such as ethanol, and phosphate buffer solutions.

Suitable suspending agents include AVICEL RC-591 (from FMC Corporation of Philadelphia, PA) and sodium alginate.

Suitable surfactants include lecithin, Polysorbate 80, and sodium lauryl sulfate, and the TWEENS from Atlas Powder Company of Wilmington, Delaware. Suitable surfactants include those disclosed in the C.T.F.A. Cosmetic Ingredient Handbook, 1992, pp. 587-592; Remington's Pharmaceutical Sciences, 15th Ed. 1975, pp. 335-337; and McCutcheon's Volume 1, Emulsifiers & Detergents, 1994, North American Edition, pp. 236-239.

### 3. EXAMPLES

### Example 1

To examine the formation of MEG-3 assemblies in more detail, ultracentrifugation monitored MEG-3 precipitates across a range of MEG-3 concentrations. MEG-3 was purified from Escherichia coli under denaturing conditions as a His-tagged fusion, as known in the art (see Smith, J. et al. eLife 5, e21337 (2016), incorporated herein by reference). Alternatively, MEG-3 could be purified under native condition, for example, as an MBP fusion followed by protease removal of MBP (see Putnam et al. Nature Structural and Molecular Biology (2019) 26:220-226, incorporated herein by reference). As expected, the proportion of MEG-3 in precipitates increased with increasing MEG-3 concentrations (FIG. 1A). The pool of soluble MEG-3 decreased sharply past the critical concentration for precipitate formation (FIG. 1B). This aggregation-like or “clustering” behavior contrasts with other non-dynamic assemblies proposed to form by LLPS followed by gelation of the dense phase.

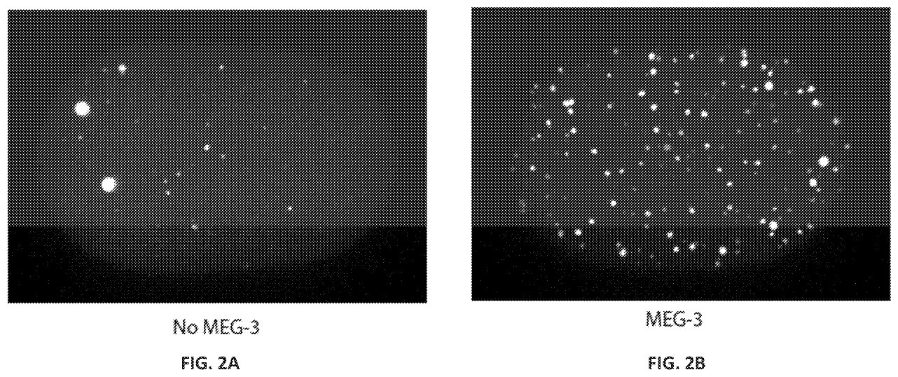

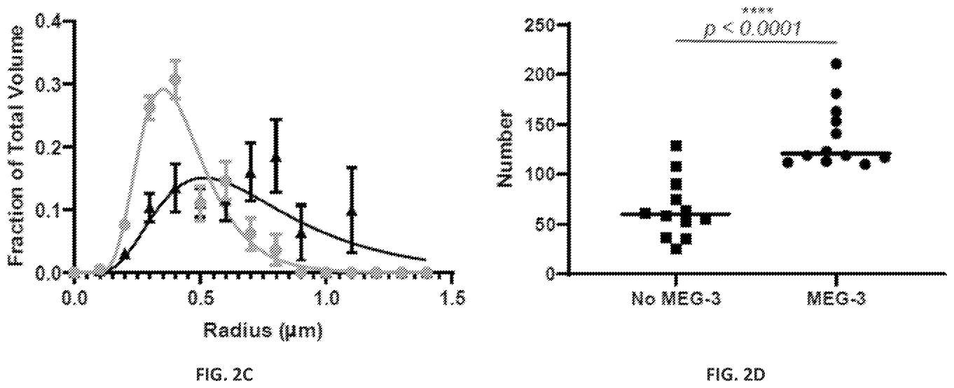

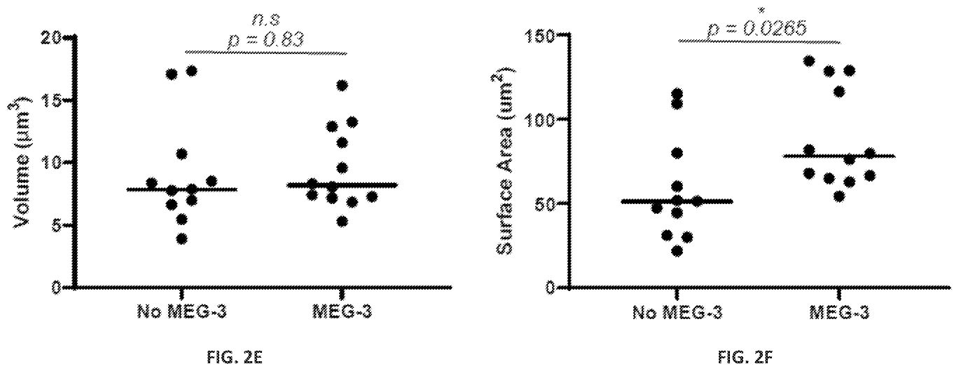

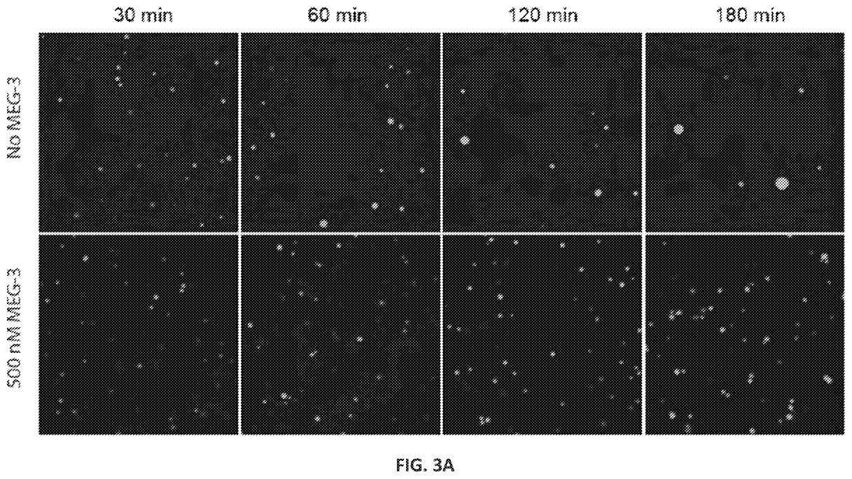

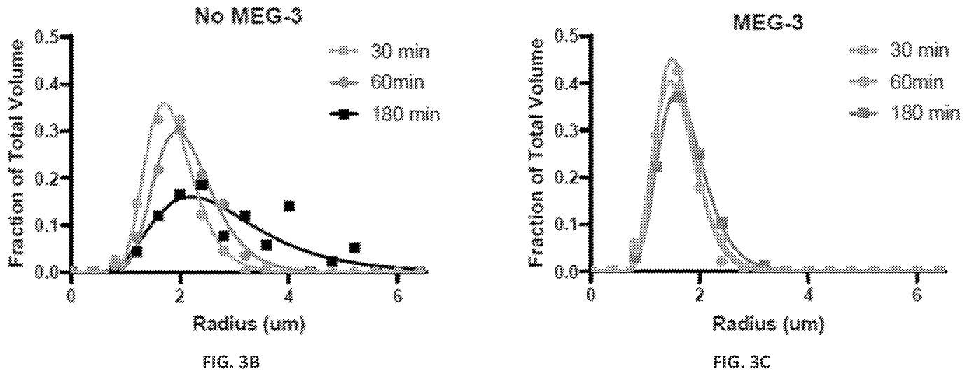

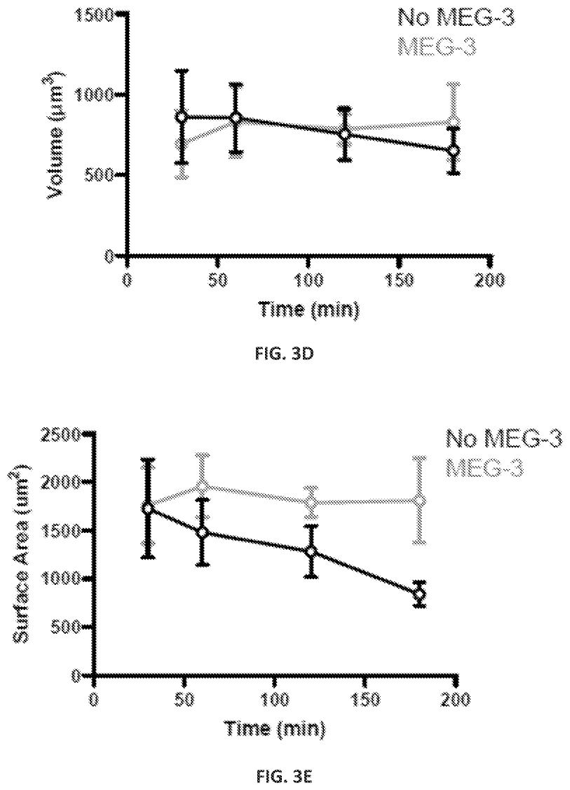

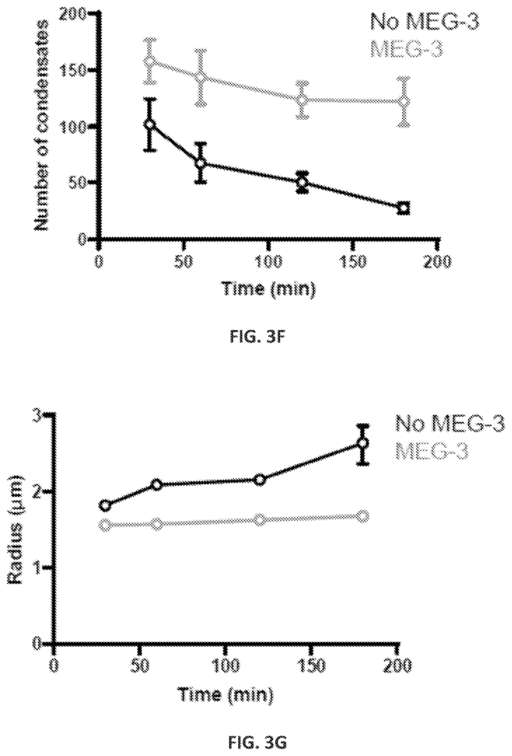

Whether MEG-3 can inhibit coarsening of a PGL-3 emulsion was tested. When placed in condensate buffer, PGL-3 initially forms a relatively homogeneous emulsion of small droplets (<2 μm radius). Over time, the PGL emulsion coarsens characterized by an increase is size and decrease of the total number of droplets (FIG. 3A-B, F, G). This progression happens without a change in the total volume of PGL-3 in droplets, confirming that PGL-3 solubility does not change over time under in vitro conditions (FIG. 3D). Addition of MEG-3 clusters to the PGL-3 emulsion blocked coarsening as droplet size and number stabilized after 30 min and remained unchanged throughout the remainder of the time-course (180 min) (FIG. 3D-G). In addition, a 3D reconstruction of the PGL emulsion using super resolution images of embryos captured in late meiosis of with the presence and absence of MEG-3 confirmed that MEG-3 prevented coarsening of PGL-3 droplets (FIG. 2).

Similar experiments with Fused in sarcoma (FUS), a DNA/RNA-binding protein, Ras GTPase-activating protein-binding protein G3BP, or DEAD box protein 3-X-chromosomal (DDX3)/LAF-1 may also show stabilization of the composition with MEG-3.

It is understood that the foregoing detailed description and accompanying examples are merely illustrative and are not to be taken as limitations upon the scope of the invention, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

What is claimed is:

1. A emulsion system comprising:
an intrinsically disordered protein selected from the group consisting of a MEG (maternal-effect germline defective) protein or a combination thereof;
a protein in need of stabilization exogenous to the intrinsically disordered protein; and
water.

2. The emulsion system of claim 1, wherein the intrinsically disordered protein comprises MEG-3.

3. The emulsion system of claim 1 or claim 2, wherein the mole or molar ratio of the protein in need of stabilization and the intrinsically disordered protein is about 2 to about 20.

4. The emulsion system of any of claims 1-3, wherein the emulsion system further comprises a single-stranded nucleic acid.

5. The emulsion system of claim 4, wherein the single-stranded nucleic acid comprises RNA.

6. The emulsion system of claim 4 or 5, wherein the single-stranded nucleic acid associates with the intrinsically disordered protein.

7. The emulsion system of any of claims 1-6, wherein the emulsion system further comprises a polyanion.

8. The emulsion system of any of claims 1-7, wherein the emulsion system comprises a water-in-water emulsion.

9. A composition comprising the emulsion system of any of claims 1-8.

10. The composition of claim 9, wherein the composition is a cosmetic composition, a pharmaceutical or nutraceutical composition, a pesticide or insecticide composition, a fertilizer, a food, a drink, or a nanomaterial.
